(19) United States
(12) Patent Application Publication
(10) Pub. No.: US 2005/0187549 A1
(43) Pub. Date: Aug. 25, 2005

(54) Removable medical implant closure

(76) Inventor: Roger P. Jackson, Prairie Village, KS (US)

Correspondence Address: LAW OFFICE OF JOHN C. MCMAHON, P.O. BOX 30069, KANSAS CITY, MO, 64112, US

(21) Appl. No.: 11/110,405

(22) Filed: Apr. 20, 2005

Related U.S. Application Data
(63) Continuation-in-part of application No. 09/588,924, filed on Jun. 6, 2000, now Pat. No. 6,884,244.

(51) Int. Cl. A61B 17/56

(52) U.S. Cl. 606/61; 606/72

(57) ABSTRACT

A closure for use in conjunction with an open receiver of a medical implant for capturing and locking a rod member in the implant includes a radially outward threaded cylindrical body sized and shaped to be threadably received between two arms of a head of the implant. The closure also includes a driving head and a coaxial removal head having a similar shape. The driving head breaks away from the body at a predetermined torque leaving the removal head. A rim stop is located near a base of the driving head to prevent a tool with a socket for gripping the driving head from inadvertently gripping the removal head during installation and over-torquing the closure. The stop is removed with the driving head, leaving the removal head that may be received by the same socket tool used for installation. The closure may also be in the form of a set screw and plug combination, with the set screw including a breakaway driving head and a removal head with a polyhedral shaped external surface.

### CROSS REFERENCE TO RELATED APPLICATION

This is a continuation-in-part of application Ser. No. 09/588,924, filed Jun. 6, 2000.

### BACKGROUND OF THE INVENTION

The present invention is directed to a closure for use in conjunction with medical implants that have open or closed receivers or heads for receiving rods and the like and, in particular, to such a closure that includes a break-off installation head and a second removal head.

Various medical implants that are used in conjunction with spinal surgery include open receivers or heads that receive rods and other elements of an overall implant system. These implants include bone screws, hooks and related parts that are variously used to produce an overall implant system. The implant system, in turn, provides support to a patient's spine to compensate for disease, injury or congenital defects.

Open headed implants or receivers normally have a pair of spaced arms that are positioned on opposite sides of a channel that receives a rod or the like for securing the implant to the rod. The open headed implants are often preferable in certain situations where it is better to lay a rod or other element into the head rather than thread a rod through a closed head. For example, where a rod must join with a large number of bone screws along a substantial length of curved spine, it is extremely difficult, if not impossible, to thread the rod through each of the bone screws and follow the curvature of the spine at the same time. Consequently, open headed elements are typically very important for use with spinal implant systems. However, open headed implants have to be effectively closed to capture the rod or rod-like member and locked in order to secure the rod member in a fixed position relative to the implant and further the closure must be removable should it be necessary to disassemble at least that portion of the overall implant system for some reason.

Plug-like closures have been provided for open headed implants in the prior art. Such prior art closures are externally threaded and are screwed into mating threads on the interior surfaces of the implant arms. Most of the prior art plug like closures have had a fairly large profile in that they extend substantially above the implant in order to have sufficient structure to both install and remove the plug or, alternatively, the implant is made taller. Both of these alternatives are undesirable, since it is preferred to have as low a profile as possible with respect to the overall system in order to have a minimal impact on the patient's body subsequent to installation. Furthermore, many of the prior art devices cannot be sufficiently tightened or torqued against the rod member so as to lock the rod from both axial and rotational movement relative to the implant. The various elements of the overall implant system are relatively small and the body can exert substantial forces on these elements, especially in situations where greater than normal forces are applied, such as accidents or the like. Slippage between the various elements can result in failure of the overall system and serious injury to a patient.

Consequently, it is desirable to be able to both lock the rod member relative to the implant with the closure by use of high torquing forces during installation with a relatively low profile subsequent to installation and yet still have sufficient structure and ability to remove the closure should it be necessary at a later time.

### SUMMARY OF THE INVENTION

A closure is provided for a medical implant. The implant may be a bone screw, hook or other element used in a spinal implant system for providing support or reconstruction to the spine, such as a transverse connector. A typical implant for use with a closure of the invention includes a receiver having a pair of spaced arms with a channel located therebetween. The channel receives a rod or other elongate structure. A closure of the invention is used to capture and fix the rod in the receiver subsequent to the receiver receiving the rod. In particular, internal surfaces of the arms of the receiver include a discontinuous guide and advancement structure, and the closure includes cooperating external guide and advancement structure so as to be rotated and driven into the receiver. Once the closure is matingly received in the receiver, the closure acts to capture the rod member.

The closure includes a break-off portion having a driving or installation head that in one embodiment has a polyhedral shaped external surface that is sized and shaped to be received in a socket of a driving tool. The closure is torqued by the driving tool acting on the driving head until a predetermined torque is achieved at which time, the break-off portion breaks away from a body of the closure. The breaking away of the driving head provides for a low profile.

The closure further includes a second driving or removal head that has a polyhedral cross section. In the illustrated embodiment, the removal head has a polyhedral cross section that is substantially the same as a polyhedral cross section of the driving head. Additionally, the closure includes structure providing a barrier, prohibiting engagement of the removal head by a socket type tool engaging the driving head. Such structure may be a projection or rim disposed between the driving head and the removal head. Thus, the driving tool cannot be accidently used to drive both heads and over-torque the closure upon installation. The projection or rim is removed with the driving head, allowing for the same driving tool to be used to mate with the removal head for rotating the closure out of the implant.

Also according to the invention, the closure may be in the form of a set screw that may be nested within a plug to form a nested closure for securing a structural element, such as a rod, within a receiver of a medical implant, such as an open-headed bone screw. The outer plug is adapted to be interferingly positioned within the opening of the receiver. The inner set screw has a break-off driving head, a removal head and a rim or other projection disposed between the driving head and the removal head. According to an aspect of the invention, when installed, only the set screw frictionally engages the rod or other structural element. The set screw equipped with a breakaway or break-off driving head, a removal head, and a projection or rim disposed therebetween may also be utilized in other types of open or closed medical implants, with or without an outer plug.

### OBJECTS AND ADVANTAGES OF THE INVENTION

Therefore, objects of the present invention include: providing a closure for use in conjunction with open ended medical implants that receive and capture a rod member, locking of the rod member with respect to the medical implant against both rotational and axial movement and removal of the closure should removal be necessary; providing such a closure having a plug body that is sized and shaped to be mateably received in threads of arms associated with the medical implant; providing such a closure or set screw therefor that includes a driving head that breaks away at a predetermined torque to provide a comparatively low profile; providing such a closure or set screw therefore that includes a removal head that remains with the closure or set screw subsequent to breakaway of the driving head; providing structure such that a socket tool utilized for torquing the driving head cannot be inadvertently engaged with the removal head to over torque the closure or set screw upon installation; and providing such a closure or set screw for such a closure and an overall system that is relatively easy to use, inexpensive to produce and especially well adapted for the intended usage thereof.

Other objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is an enlarged and perspective view of a bone screw closure according to the present invention.

FIG. 2 is an enlarged front elevational view of the closure of FIG. 1.

FIG. 3 is an enlarged top plan view of the closure of FIG. 1.

FIG. 4 is an enlarged bottom plan view of the closure of FIG. 1.

FIG. 5 is a reduced exploded perspective view of an open headed polyaxial bone screw assembly including the closure of FIG. 1.

FIG. 6 is a fragmentary side elevational view of the bone screw assembly of FIG. 5, shown with a rod in cross section and shown with an installation/removal tool.

FIG. 7 is a fragmentary and enlarged view of the assembly and the installation/removal tool of FIG. 6 with portions broken away to show the detail thereof.

FIG. 8 is an exploded and fragmentary side elevational view similar to FIG. 6, but without the installation/removal tool and showing a break-off head removed from the closure.

FIG. 9 is an exploded and fragmentary side elevational view similar to FIG. 8, showing the bone screw assembly with the installation/removal tool.

FIG. 10 is an enlarged cross-sectional view of the bone screw assembly and installation/removal tool of FIG. 9, showing the tool engaged with the closure.

FIG. 11 is a top plan view of the bone screw assembly of FIG. 9.

FIG. 12 is an enlarged and perspective view of a set screw embodiment according to the present invention.

FIG. 13 is an enlarged front elevational view of the set screw of FIG. 12.

FIG. 14 is an enlarged top plan view of the set screw of FIG. 12.

FIG. 15 is an enlarged bottom plan view of the set screw of FIG. 12.

FIG. 16 is an exploded perspective view of an alternative embodiment of an open headed bone screw assembly including a closure having the set screw of FIG. 12.

FIG. 17 is a fragmentary side elevational view of the bone screw assembly of FIG. 16, shown with a rod in cross section and shown with a set screw installation/removal tool.

FIG. 18 is a fragmentary and enlarged view of the assembly and tool of FIG. 17 with portions broken away to show the detail thereof.

FIG. 19 is an exploded and fragmentary side elevational view similar to FIG. 18, but without the installation/removal tool and showing a break-off head removed from the set screw.

FIG. 20 is an exploded and fragmentary side elevational view similar to FIG. 19, showing the bone screw assembly with the installation/removal tool.

FIG. 21 is an enlarged and fragmentary cross-sectional view of the bone screw assembly and installation/removal tool of FIG. 20, showing the tool engaged with the set screw.

FIG. 22 is a top plan view of the bone screw assembly of FIG. 20.

### DETAILED DESCRIPTION OF THE ILLUSTRATED EMBODIMENT

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

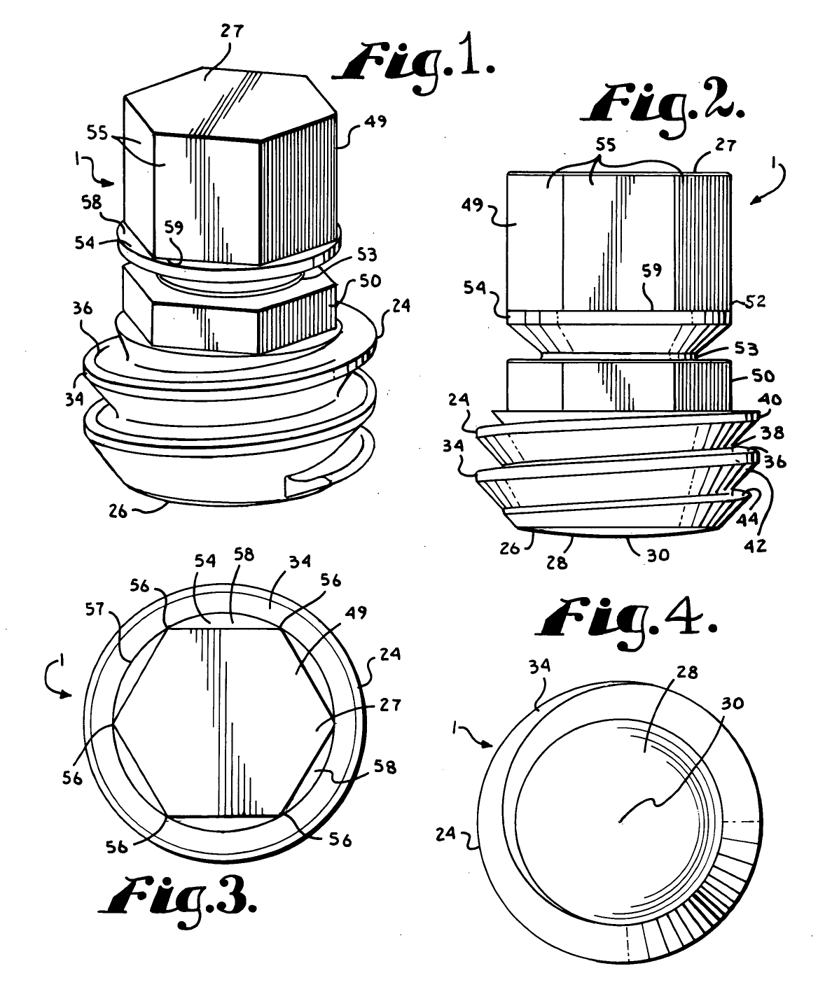

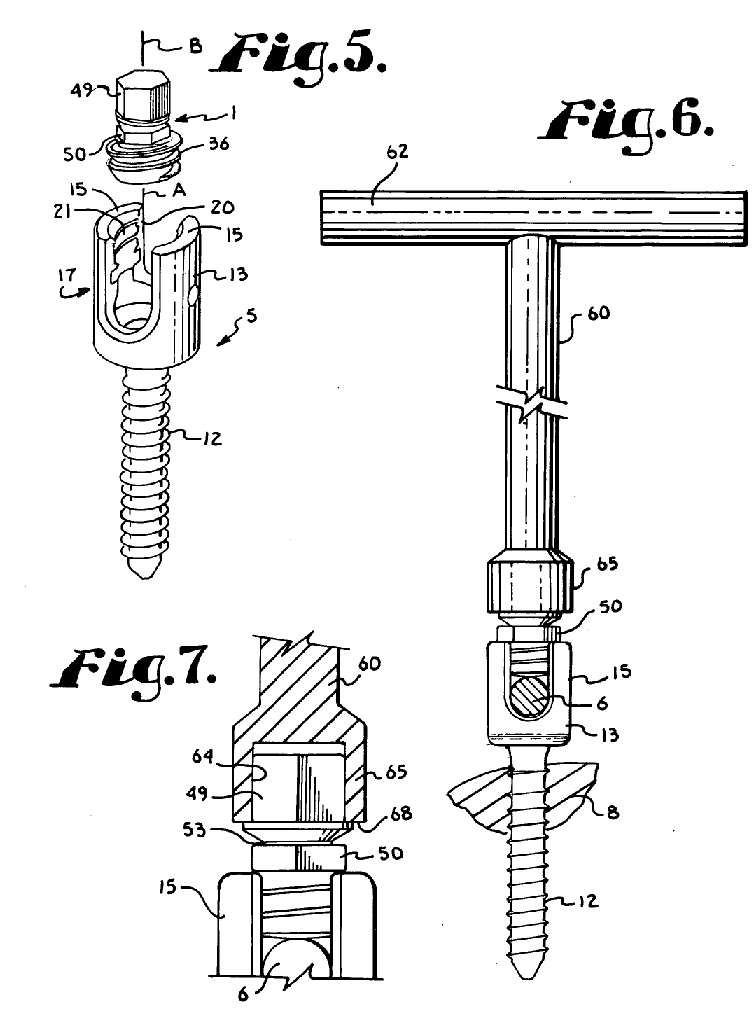

With reference to FIGS. 1-11, the reference numeral 1 generally designates a closure according to the present invention. The closure 1 is shown utilized to close a top of a polyaxial medical implant bone screw, generally 5, and capture an elongate member or rod 6, the bone screw 5 and the rod 6 being operably incorporated in an overall spinal implant system for correcting degenerative conditions, deformities, injuries, or defects to the spinal column of the patient. In use the bone screw 5 is inserted into a vertebra 8. It is noted that any reference to the words top, bottom, up and down, and the like, in this application refers to the alignment shown in the various drawings, as well as the normal connotations applied to such devices, and is not intended to restrict the positioning of bone screws and closures in actual use.

With reference to FIG. 5, the polyaxial bone screw 5 includes a shank 12 pivotally attached to an open receiver or head 13. The shank 12 is threaded and has a central axis of rotation A. The receiver 13 has a pair of spaced and generally parallel arms 15 that form a channel 17 therebetween that is open at the distal ends of the arms 15. The arms 15 each include radially inward or interior surfaces 20 that have a discontinuous guide and advancement structure 21 mateable with cooperating structure on the closure 1 described more fully below.

The shank 12 and the receiver 13 may be attached in a variety of ways. For example, a spline capture connection described in U.S. Pat. No. 6,716,214, and incorporated by reference herein, may be utilized in which the bone screw shank includes a capture structure mateable with a retaining structure disposed within the receiver. The retaining structure includes a partially hemispherical surface that is slidingly mateable with a cooperating inner surface of the receiver, allowing for a wide range of pivotal movement between the shank and the receiver. Other types of capture connections may also be used including, but not limited to, threaded connections, frictional connections utilizing frusto-conical or polyhedral capture structures, integral top or downloadable shanks, and the like. Furthermore, although the closure 1 of the present invention is illustrated with the polyaxial bone screw 5 having an open receiver or head 13, it foreseen that the closure 1 may be used in conjunction with any type of medical implant having a similar type of open head, including monoaxial bone screws, hooks and the like used in spinal surgery.

With reference to FIGS. 6-11, the rod 6 is an elongate, often curved, rod or elongate rod-like member that generally extends between multiple bone screws 5 of the type shown here or other elements of a spinal system. It is also foreseen that the rod 6 could be a connector between two laterally spaced elements of the overall system and similar structures that are elongate or have rod-like portions that can be placed within the channel 17. The illustrated rod 6 is circular in cross section and has a smooth external surface; however in accordance with the invention it is foreseen that rods having other types of cross section and having rough or knurled external surfaces could be utilized. During use, the rod 6 is located or positioned within the bone screw channel 17 and secured in place therein by the closure 1.

With reference to FIGS. 1-4, the closure 1 includes a body 24 that has a generally cylindrical or plug shape with a substantially circular horizontal cross section and a central axis of rotation B. Located at a lead or bottom end 26 of the closure 1 opposite a substantially flat top surface 27 is a convex shaped region or dome 28 that projects outwardly from the body 24 along the axis B (downwardly in FIG. 2) and has an apex 30 where the dome 28 intersects the axis B. In the illustrated embodiment, the dome 28 covers the entire bottom end 26 of the closure 1. It is foreseen that domes in accordance with the invention may cover more or less of the bottom surface and may vary in radius of generation or curvature. It is preferred that the dome 28 be smooth and convex where the axis B intersects with the dome 48 and not pointed. However, it is foreseen that a bottom surface of the closure body may be substantially flat and include a small dome having a central point for gripping and abrading a rod surface. It is also foreseen that in certain embodiments, a domed bottom may have an apical point or may be at least partially covered with knurling or the like to provide additional gripping during usage. In addition, the bottom surface may be flat and may have a central point and a lateral rim.

Located on the cylindrical closure body 24 is a guide and advancement structure 34 mateable with the guide and advancement structure 21 on the arms 15 of the bone screw receiver 13. In the illustrated embodiment, the guide and advancement structure 34 is a helically wound reverse angle thread form 36. The guide and advancement structure 34 acts cooperatively, as described below with the bone screw receiver 13 to allow the closure 1 to be inserted into and rotated relative to the bone screw receiver 13 and to guide and advance the closure 1 along the axis B as the closure 1 is rotated clockwise or to reverse direction when rotated counterclockwise. The guide and advancement structure 34 resists splaying in the bone screw receiver 13 as forces applied to the closure 1 are conveyed by the reverse angle thread form 36 during application of clockwise rotational torque into a downward axial force and inwardly directed radial force. It is foreseen that other types of guide and advancement structure could be utilized. For example, a buttress thread form, a square thread form or some other type of structure such as a flange form may be effectively used which theoretically has little or no radially outward directed forces, especially if the arms are thickened to resist splaying.

The thread form 36 has a root 38 and a crest 40. Further, the thread form 36 has a lead surface 42 and a trailing surface 44 (described relative to the position thereof during insertion of the closure 1 into the bone screw receiver 13). In a reverse angle thread, the trailing surface 44 from the root 38 to the crest 40 extends at an angle rearwardly from a perpendicular line relative to the axis B. Normally, the trailing surface 44 is at an angle between 1 and 20 degrees relative to such a perpendicular.

The closure 1 also includes a driving or installation break-off head 49 and a removal head 50 that are coaxially attached to the body 24. The removal head 50 is located between the body 24 and the driving head 49 and disposed near the guide and advancement structure 34. The driving head 49 is disposed on a break-off portion 52 that is secured to the body 24 at a breakaway region 53 adjacent the removal head 50 and is designed to break away from the remainder of the closure 1 subsequent to a predetermined torque being applied to the driving head 49, such as 100 inch pounds, during installation of the closure 1 into the bone screw 5. A barrier in the form of a cylindrical rim stop 54 is disposed on the break-off portion 52 between the breakaway region 53 and the driving head 49. The illustrated rim stop 54 is adjacent to the driving head 49. As illustrated in FIG. 3, the driving head 49 top surface 27, that is perpendicular to the axis of rotation B, is hexagonal and is formed by six flat faces 55 that are joined together in a hexagonal pattern. Each of the faces 55 runs parallel to the axis of rotation B. Together, the faces 55 form a hexagonal driving feature adapted for use with a socket type driving tool, described more fully below. The illustrated rim stop 54 is substantially circular in cross-section and concentric with the driving head 49. As shown in FIG. 3, edges 56 formed by the six flat faces 55 of the driving head 49 define in part an outer edge or circumference 57 of the rim stop 54, with the rim stop 54 forming a discontinuous annular surface 58 between each of the edges 56 at a lower end or base 59 of the driving head 49.

An installation and removal tool 60 is provided for driving and torquing the driving head 49 and if needed or desired, driving the removal head 50. The installation/removal tool 60 includes a gripable handle 62 that allows a user to rotate the tool 60, and in particular a hexagonal socket 64 disposed at a lower end 65 of the tool 60. The socket 64 is shaped and sized to snugly receive both the driving head 49, as shown in FIGS. 6 and 7 and the removal head 50 as shown in FIG. 10. In use, the discontinuous annular surface 58 of the rim stop 54 abuts against a bottom surface 68 of the tool 60 when the socket 64 engages the driving head 49, providing a barrier so that the installation/removal tool 60 cannot inadvertently grip the removal head 50 when installing the closure 1 and thereby produce too much torque by bypassing the torque limitation associated with the break-off driving head 49.

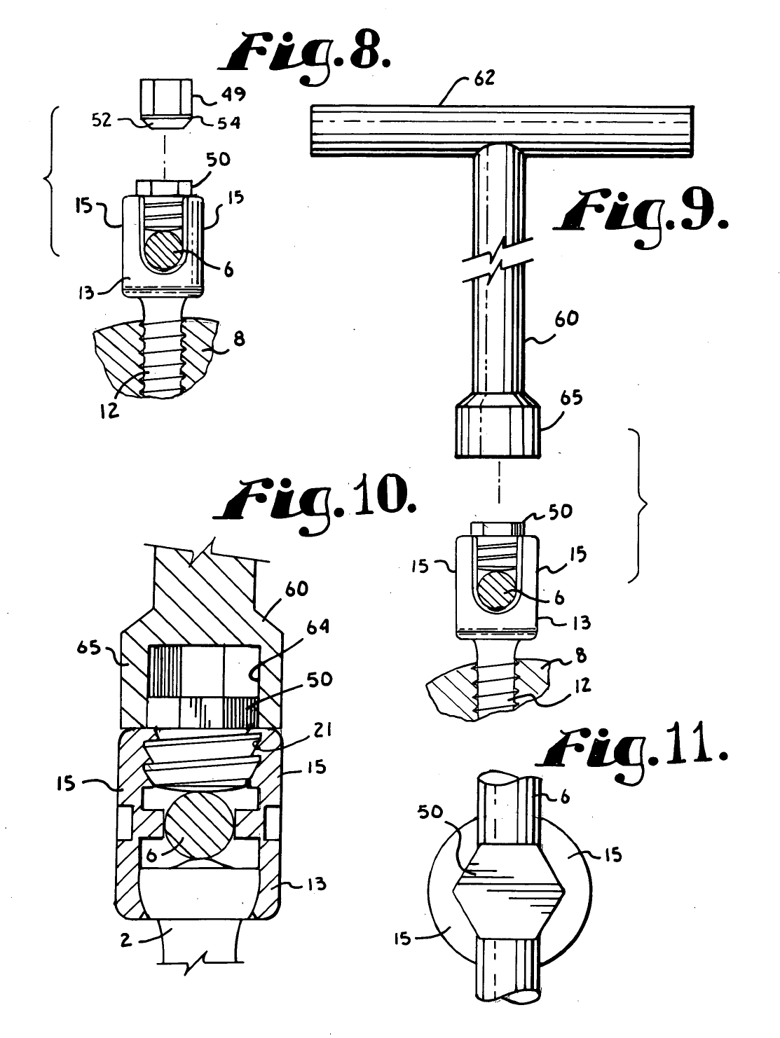

FIG. 8 shows the driving head 49 and the rim stop 54 broken away from the body 24. Although the illustrated rim stop 54 is substantially cylindrical and therefore the circumference 57 is circular, it is foreseen that the rim stop may be of a variety of other geometries that extend outwardly from one or more faces 55 of the driving head 49 so that the tool 60 abuts the stop and does not engage with the removal head 50 when engaged with the driving head 49.

The illustrated removal head 50 has a hexagonal cross-section substantially identical in size and shape to the driving head 49 so that the same tool 60 may be used for both installation and removal. In the illustrated embodiment the removal head 50 is integral with the closure body 24. If, subsequent to installation, it is necessary or desirable to remove the closure 1, the tool 60 may be used as illustrated in FIG. 10. The socket 64 is snugly mateable with the removal head 50 to allow the closure 1 to be rotated and removed from the bone screw receiver 13. Because removal usually takes less torque in comparison to installation, the removal head structure does not need to be as tall as the head 49. It is also foreseen that if desired, the head 50 could be smaller than the head 49, requiring a different removal tool (not shown) with a smaller socket.

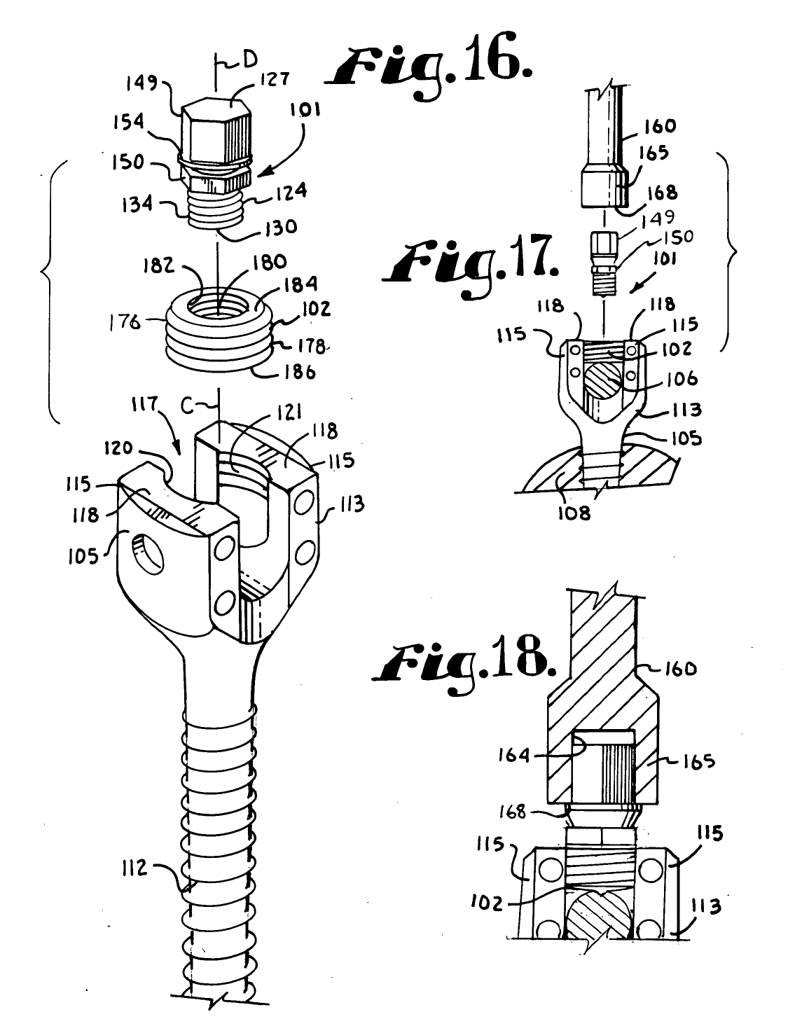

It is foreseen that a medical implant closure according to the invention may include a removal head and driving head combination similar to the embodiments shown in FIGS. 1-22, but with the driving feature of the driving head being an imprint or other concave, internal driving formation formed in a top surface of the driving head. In such an embodiment, the removal head would be disposed on or adjacent a body or plug portion of the closure, and the driving feature would be spaced from the removal head and disposed on a break-off portion of the closure. Thus, the removal head having a radially outwardly extending driving surface would be positioned axially between the closure body and the internal driving feature. However, the removal head could not be accessed until the break-off portion is first removed, utilizing the external or internal driving feature for the removable portion of the closure. It is also foreseen that in such an embodiment, the imprint or other internal driving formation in the driving head may be sized such that the same driving tool could be utilized to both rotate the internal driving head feature in the break-off portion, and subsequently the external removal head feature, after the break-off portion is removed.

In use, the bone screw 5 is implanted into a vertebra 8. Eventually the rod 6 is positioned in the channel 17 of the bone screw receiver 13 in generally perpendicular relationship to the shank 12. With reference to FIGS. 5-8, the installation/removal tool 60 is utilized to install the closure 1 into a bone screw receiver 13 by engaging and rotating the driving head 49 with the socket 64, during which installation the driving head 49 breaks from the body 24 of the closure 1 at a predetermined torque so as to tightly snug the dome 28 of the closure 1 against the rod 6 and lock the rod 6 in position relative to the bone screw 5. FIGS. 8 and 11 illustrate the fully installed closure 1 in a receiver 13 and capturing a rod 6 therewithin. With reference to FIGS. 9-10, if removal of the closure 1 is required, the installation/removal tool 60 is again utilized to rotate and remove the closure body 24 by engaging the removal head 50 with the socket 64 and rotating the closure body 24 out of the bone screw receiver 13.

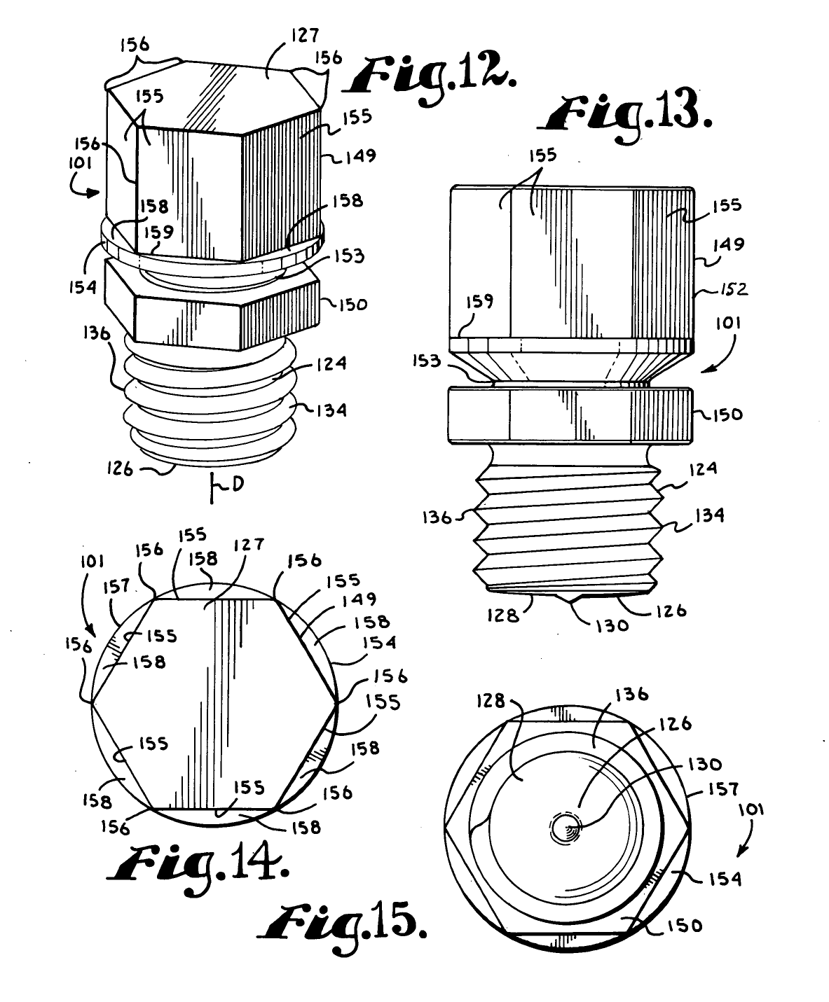

With reference to FIGS. 12-22, the reference numeral 101 generally represents a second or alternative embodiment for a set screw in combination with a closure plug according to the present invention. The set screw 101 is shown with a cooperating plug 102 utilized to close a top of a medical implant bone screw, 105, and capture an elongate member or rod 106, the bone screw 105 and the rod 106 being operably incorporated in an overall spinal implant system for correcting deformities, injuries, or defects to the spinal column of the patient. In use the bone screw 105 is inserted into a vertebra 108.

With reference to FIG. 16, the bone screw 105 includes a shank 112 integral with an open receiver or head 113. The shank 112 is threaded and has a central axis of rotation C. The receiver 113 has a pair of spaced and generally parallel arms 115 that form a channel 117 therebetween that is open at a top or distal end surface 118 of each of the arms. The top surfaces 118 of the arms 115 are coplanar. The arms 115 each include radially inward or interior surfaces 120 that have a discontinuous guide and advancement structure or thread 121 mateable with cooperating structure on the closure plug 102 as described more fully below.

In the illustrated embodiment, the shank 112 and the receiver 113 are integral, but it is foreseen that a polyaxial screw may also be utilized with the set screw 101 similar to the polyaxial bone screws previously described herein with respect to the closure 1, and incorporated herein by reference. Also, it is foreseen that the set screw 101 may be utilized with a variety of open and closed bone screw implants including hooks, transverse connectors, and the like.

The rod 106 is an elongate, often curved, rod or elongate rod-like member that generally extends between multiple bone screws 105 of the type shown here or other elements of a spinal system. It is also foreseen that the rod 106 could be a connector between two laterally spaced elements of the overall system and similar structures that are elongate or have rod-like portions that can be placed within the channel 117. The illustrated rod 106 is circular in cross section and has a smooth external surface, however in accordance with the invention it is foreseen that rods having other types of cross section and having rough or knurled external surfaces could be utilized. During use, the rod 106 is located or positioned within the bone screw channel 117 and secured in place therein by the plug 102 and the set screw 101. However, it is noted that in this embodiment only the set screw 101 frictionally engages the rod. The plug 102, when fully installed, contacts only the bone screw arms 115 and the set screw 101. In other embodiments, the plug could also contact the rod.

With reference to FIGS. 12-15, the set screw 101 includes a body 124 that has a generally cylindrical or plug shape with a substantially circular horizontal cross section and a central axis of rotation D. Located at a lead or bottom end 126 of the set screw 101 opposite a substantially flat top surface 127 is a flat or slightly convex shaped region 128 that includes a point or projection 130 extending outwardly from the body 124 along the axis D (downwardly in FIG. 13). The point 130 is sized and shaped for gripping and penetrating the rod 106. It is also foreseen that in certain embodiments, a domed bottom similar to the dome 28 previously described herein with respect to the closure 1 may be disposed at the region 128 and extend outwardly from the body 124 along the axis D.

Located on the cylindrical body 124 is a guide and advancement structure 134 mateable with an inner guide and advancement structure on the plug 102 to be described more fully below. In the illustrated embodiment, the guide and advancement structure 134 is a helically wound V-shaped thread form 136. The guide and advancement structure 134 acts cooperatively, as described below with the plug 102 to allow the set screw 101 to be inserted into and rotated relative to the plug 102. It is foreseen that other types of guide and advancement structure could be utilized in lieu of the thread form 136, including a reverse angle thread, a buttress thread form, a square thread form or other type of flange form.

The set screw 101 also includes a driving or installation break-off head 149 and a removal head 150 that are coaxially attached to the body 124. The removal head 150 is located between the body 124 and the driving head 149 and disposed near the guide and advancement structure 134. The driving head 149 is disposed on a break-off portion 152 that is secured to the body 124 at a breakaway region 153 adjacent the removal head 150. The break-off portion 152 is designed to break away from the remainder of the set screw 101 subsequent to a predetermined torque being applied to the driving head 149, such as 100 inch pounds, during installation of the set screw 101 into the plug 102. A cylindrical barrier or rim stop 154 is disposed on the break-off portion 152 between the breakaway region 153 and the driving head 149. The illustrated rim stop 154 is adjacent to the driving head 149. As illustrated in FIG. 14, the driving head 149 top surface 127, that is perpendicular to the axis of rotation B, is hexagonal and is formed by six flat faces 155 that are joined together in a hexagonal pattern. Each of the faces 155 runs parallel to the axis of rotation D. The illustrated rim stop 154 is substantially circular in cross-section and concentric with the driving head 149. As shown in FIG. 14, edges 156 formed by the six flat faces 155 of the driving head 149 define in part an outer edge or circumference 157 of the rim stop 154, with the rim stop 154 forming a discontinuous, partially annular abutment surface 158 between each of the edges 156 at a lower end or base 159 of the driving head 149.

An installation and removal tool 160 is provided for driving and torquing the driving head 149 and if needed or desired, driving the removal head 150. The installation/removal tool 160 includes a gripable handle 162 that allows a user to rotate the tool 160, and in particular a hexagonal socket 164 disposed at a lower end 165 of the tool 160. The socket 164 is shaped and sized to snugly receive both the driving head 149, as shown in FIGS. 17 and 18 and the removal head 150 as shown in FIGS. 20 and 21. In use, the discontinuous annular surface 158 of the rim stop 154 abuts against a bottom surface 168 of the tool 160 when the socket 164 engages the driving head 149, providing a barrier so that the installation/removal tool 160 cannot inadvertently grip the removal head 150 when installing the set screw 101 and thereby produce too much torque by bypassing the torque limitation associated with the break-off driving head 149.

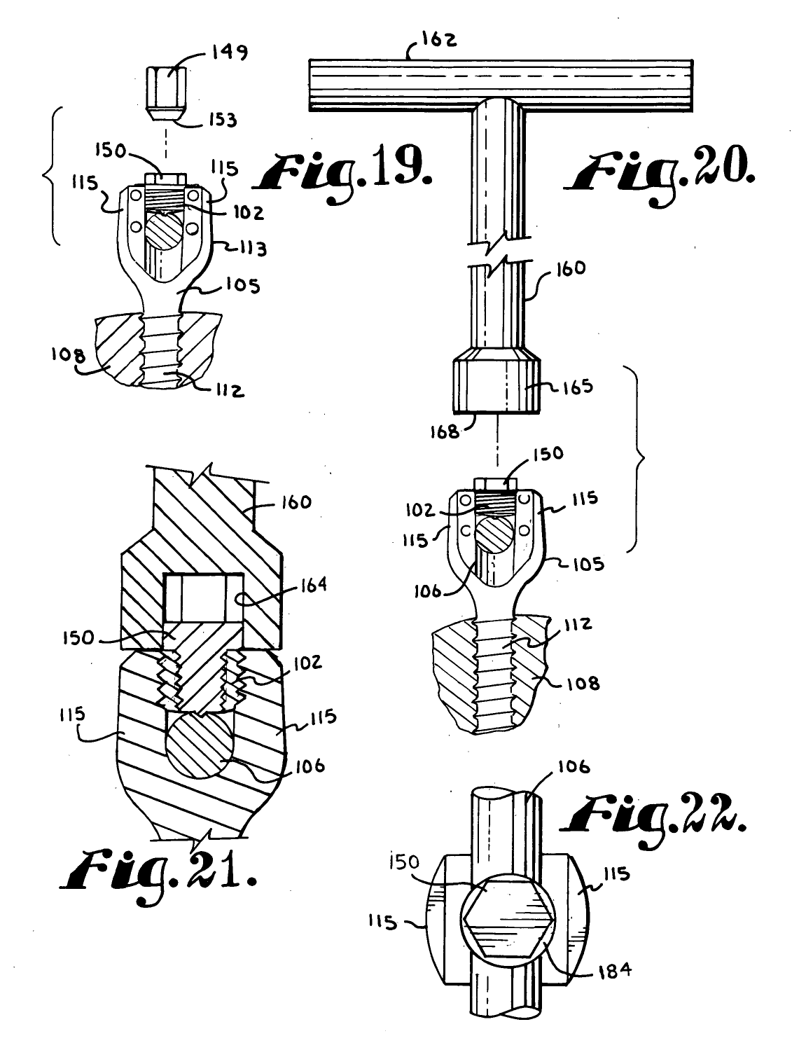

FIG. 19 shows the driving head 149 and the rim stop 154 broken away from the set screw body 124. Although the illustrated rim stop 154 is substantially cylindrical and therefore the circumference 157 is circular, it is foreseen that the rim stop may be of a variety of other geometries that extend outwardly from one or more faces 155 or face corners of the driving head 149 so that the tool 160 abuts the stop and does not engage with the removal head 150 when engaged with the driving head 149.

The illustrated removal head 150 has a hexagonal cross-section substantially identical in size and shape to the driving head 149 so that the same tool 160 may be used for both installation and removal. In the illustrated embodiment the removal head 150 is integral with the closure body 124. If, subsequent to installation, it is necessary or desirable to remove the set screw 101, the tool 160 may be used as illustrated in FIG. 21. The socket 164 is snugly mateable with the removal head 150 to allow the set screw 101 to be rotated and removed from the plug 102. Because removal usually takes less torque in comparison to installation, it is foreseen that if desired, the head 150 can be shorter and even smaller than the head 149, requiring a different removal tool (not shown) with a smaller socket.

With reference to FIG. 16, the plug 102 includes a radial outer surface 176 having a guide and advancement structure thereon, illustrated as a V-shaped thread 178. The thread 178 is sized and shaped to mate with the guide and advancement structure 121 disposed on the interior surfaces 120 of the bone screw receiver 113. Although a simple threaded connection is shown in the drawings, any type of guide and advancement structure may be used for the guide and advancement structures 121 and 178, including but not limited to, V-type threads, buttress threads, reverse angle threads, and square threads as well as flange forms.

The plug 102 also includes a central bore 180 having a guide and advancement structure 182 thereon for receiving and mating with the guide and advancement structure 134 of the set screw 101. As with the guide and advancement structures 121 and 178, although a simple threaded structure is shown, the guide and advancement structures 134 and 182 may be of a variety of helical fasteners, including but not limited to, V-type threads, buttress threads, reverse angle threads, and square threads.

The plug 102 further includes substantially planar and parallel upper and lower surfaces 184 and 186, respectively. Preferably, the plug upper surface 184 is positioned below or even with the top 118 of the bone screw receiver 113 subsequent to installation therein to provide a low profile. Preferably, the plug 102 is sized and shaped such that when fully installed in the bone screw receiver 113, with the upper surface 184 flush or below the top 118 of the receiver 113, and in some embodiments the lower surface 186 is spaced from, and thus does not frictionally engage or otherwise contact the rod 106.

An associated plug closure driving tool (not shown) preferably includes a T-shaped handle, shaft and terminus opposite the handle that is threaded for mating engagement with the guide and advancement structure 182 of the plug central bore 180. An example of such a driving tool is disclosed in U.S. Pat. No. 6,224,598, incorporated by reference herein.

In use, the bone screw 105 is implanted into a vertebra 108. Eventually the rod 106 is positioned in the channel 117 of the bone screw receiver 113 in generally perpendicular relationship to the shank 112. With reference to FIGS. 16 and 17, the plug 102 is threaded on the terminus of a plug closure driving tool (not shown) and then coaxially aligned with the bone screw 105, mating the guide and advancement structure 178 with the guide and advancement structure 121 and rotating the plug 102 in a clock-wise direction into the receiver 113. The plug 102 is preferably rotated and advanced into the receiver 113 until the top surface 184 is flush or slightly below the top surface 118 of the bone screw receiver 113, snugging the plug 102 into the arms 115, but not up against the rod 106.

With reference to FIGS. 17-19, the installation/removal tool 160 is utilized to install the set screw 101 into the plug 102 by engaging and rotating the driving head 149 in a clockwise direction utilizing the socket 164, during which installation the driving head 149 breaks from the body 124 of the set screw 101 at a predetermined torque so as to tightly engage the set screw projection or point 130 against the rod 106 and thus lock the rod 106 in position relative to the bone screw 105. FIGS. 19 and 22 illustrate the fully installed set screw 101 and plug 102 in a receiver 113 and capturing a rod 106 therewithin.

With reference to FIGS. 20 and 21, if removal of the set screw 101 is required, the installation/removal tool 160 is again utilized to rotate and remove the set screw body 124 by engaging the removal head 130 with the socket 164 and rotating the closure body 124 in a counter-clockwise direction out of the plug 102. If removal of the plug 102 is required or desired, the terminus of the plug closure driving tool (not shown) may be inserted into the threaded central bore 180 of the plug 102 and the tool rotated counter-clockwise until the plug 102 is either loosened sufficiently to permit adjustment of the rod 106 or removed entirely. The plug 102 is not torqued tightly like the set screw 101 and thus is removed comparatively easily.

It is to be understood that while certain forms of the present invention have been illustrated and described herein, it is not to be limited to the specific forms or arrangement of parts described and shown.

1. A medical implant closure comprising:
a) a substantially cylindrical body having a radially extending outward surface with a guide and advancement structure thereon;
b) a break-off portion attached to the body, the break-off portion having a first driving head with a first polyhedral radially outwardly extending driving surface and adapted to receive a socket type driving tool for rotating and torquing the closure in a medical implant until a preselected torque occurs at which time the break-off portion breaks away from the body;
c) a second driving head with a second polyhedral radially outwardly extending driving surface and adapted to receive a socket type driving tool for removal of the closure from the medical implant; and
d) a radially extending structure on the break-off portion prohibiting engagement of the second driving head by a socket type driving tool while engaged with the first driving head.

2. The closure of claim 1 wherein the radially extending structure is a projection extending from near a base of the first driving head.

3. The closure of claim 2 wherein the projection is a rim.

4. The closure of claim 1 wherein the second driving head is axially centered and positioned between the body and the break-off portion.

5. The closure of claim 1 wherein the first polyhedral radially outwardly extending driving surface is substantially identical in cross-section to the second polyhedral radially outwardly extending driving surface.

6. The closure of claim 5 wherein the radially extending structure is a rim surrounding a base of the first polyhedral radially outwardly extending driving surface.

7. The closure of claim 1 wherein the body includes a dome shaped base adapted for frictional engagement with a rod disposed in the medical implant.

8. A medical implant closure comprising:
a) a substantially cylindrical body having an axis of rotation and a radially extending outward surface with a guide and advancement structure thereon;
b) a break-off portion attached to the body, the break-off portion having a first polyhedral driving head with a plurality of radially outwardly extending driving faces and a width dimension running perpendicular to and extending through the axis of rotation and measured between a first pair of opposed driving faces;
c) a second polyhedral driving head with a plurality of radially outwardly driving faces and a second width running perpendicular to and extending through the axis of rotation and measured between a second pair of opposed driving faces; and
d) a structure on the break-off portion having a third width running perpendicular to and extending through the axis of rotation, the third width being greater than the second width.

9. The closure of claim 8 wherein the first width is substantially equal to the second width.

10. The closure of claim 8 wherein the structure is a projection extending from near a base of the first driving head.

11. The closure of claim 10 wherein the projection is a rim.

12. The closure of claim 8 wherein the body includes a dome shaped base adapted for frictional engagement with a rod disposed in the medical implant.

13. A medical implant closure comprising:
a) a substantially cylindrical body having an axis of rotation and a radially outward surface having a guide and advancement structure adapted for mating with a cooperating guide and advancement structure on an inner surface of a medical implant;
b) a break-off driving head;
c) a removal head; and
d) a radially extending projection disposed between the break-off head and the removal head.

14. The closure of claim 13 wherein the radially extending projection is a rim.

15. The closure of claim 13 wherein the driving head is joined to the body at a breakaway region adapted to break away from the body when a preselected torque is applied to the driving head.

16. The closure of claim 13 wherein the driving head and the removal head have substantially the same polyhedral shape.

17. A medical implant system comprising:
a) an open receiver formed by a pair of spaced arms defining a channel therebetween sized and shaped to receive a rod member, the arms having an interior discontinuous first guide and advancement structure thereon; and
b) a closure member comprising:
i) a substantially cylindrical body having a radially extending outward surface with a second guide and advancement structure thereon;
ii) a break-off portion attached to the body, the break-off portion having a first driving head with a first polyhedral radially outwardly extending driving surface;
iii) a second driving head with a second polyhedral radially outwardly extending driving surface; and
iv) a radially extending structure on the break-off portion prohibiting engagement of the second driving head by a socket type driving tool while engaged with the first driving head.

18. The system of claim 17 wherein the first guide and advancement structure is mateable with the second guide and advancement structure.

19. The system of claim 18 wherein the first and second guide and advancement structures each have an anti-splay contour.

20. The system of claim 17 wherein the radially extending structure is a projection extending from near a base of the first driving head.

21. The system of claim 20 wherein the projection is a rim.

22. The system of claim 17 wherein the second driving head is axially centered and positioned between the body and the break-off portion.

23. The system of claim 17 wherein the first polyhedral radially outwardly extending driving surface is substantially identical in cross-section to the second polyhedral radially outwardly extending driving surface.

24. The system of claim 23 wherein the radially extending structure is a rim surrounding a base of the first polyhedral radially outwardly extending driving surface.

25. The system of claim 17 wherein the body includes a dome shaped base adapted for frictional engagement with a rod disposed in the medical implant.

26. The system of claim 17 wherein the closure is a set screw and further comprising a plug cooperating with the set screw, the plug having an outer surface with a third guide and advancement structure thereon and an inner surface having a fourth guide and advancement structure thereon, the third guide and advancement structure mateable with the first guide and advancement structure and the fourth guide and advancement structure mateable with the second guide and advancement structure.

27. The system of claim 26 wherein the first and third guide and advancement structures are helical threads.

28. The system of claim 26 wherein the second and fourth guide and advancement structures are helical threads.

29. The system of claim 26 wherein the set screw body includes a dome shaped base adapted for frictional engagement with a rod disposed in the medical implant.

30. The system of claim 29 wherein the plug has a substantially planar bottom surface, the bottom surface spaced from the rod when the dome shaped base of the body is in frictional engagement with the rod.

31. A medical implant closure comprising:
a) a substantially cylindrical body having a radially extending outward surface with a guide and advancement structure thereon;
b) a break-off portion attached to the body, the break-off portion having a driving feature; and
c) a removal head axially located between the body and the break-off portion, the removal head having a polyhedral radially outwardly extending driving surface.
